(19) United States
(12) Patent Application Publication
(10) Pub. No.: US 2025/0107926 A1
(43) Pub. Date: Apr. 3, 2025

(54) BREAST MASSAGER

(71) Applicant: Shenzhen Lute Jiacheng Supply Chain Management Co., Ltd., Shenzhen (CN)

(72) Inventors: Rongjun LI, Shenzhen (CN); Zhen ZENG, Shenzhen (CN)

(73) Assignee: Shenzhen Lute Jiacheng Supply Chain Management Co., Ltd., Shenzhen (CN)

(21) Appl. No.: 18/501,341

(22) Filed: Nov. 3, 2023

(30) Foreign Application Priority Data
Sep. 28, 2023 (CN) 202322687789.X

(51) Int. Cl. A61F 7/10; A61F 7/00; A61F 7/02; A61H 7/00; A61H 23/02; A61N 1/36

(52) U.S. Cl. CPC A61F 7/10; A61H 7/007; A61H 23/02; A61N 1/36014; A61F 2007/0021; A61F 2007/0071; A61F 2007/0075; A61F 2007/0093; A61F 2007/0282; A61H 2201/0214; A61H 2205/082

(57) ABSTRACT

Disclosed is a breast massager, including: a housing having a surface, a cold compress component, at least one massage component, and a control module; a cold compress area is provided on the surface of the housing; the cold compress component is provided in the housing and configured to provide cooling to the cold compress area; the at least one massage component is provided in the housing and configured to massage breasts; and the control module is provided in the housing and electrically connected to the cold compress component and the massage component respectively.

### CROSS-REFERENCE TO RELATED APPLICATIONS

This application claims priority to Chinese Patent Application No. 202322687789.X, filed on Sep. 28, 2023, the entire contents of which are incorporated herein by reference.

### TECHNICAL FIELD

The present application relates to the technical field of health care equipment, and in particular to a breast massager.

### BACKGROUND

The main function of the existing breast massager is vibration massage to promote lactation and milk discharge, and solve problems such as milk stasis and mastitis for breastfeeding mothers. However, it cannot solve physiological breast congestion, swelling and other problems. At this time, breastfeeding mothers during pregnancy need to use cold compresses for breast care. The main material of cold compresses is gel, which needs to be frozen in the refrigerator before use, which is cumbersome to operate and wastes time.

### SUMMARY

The main purpose of the present application is to provide a breast massager which can perform cold compress care on breasts and is easy and quick to operate, thereby improving the user's experience.

In order to achieve the above purpose, the present application provides a breast massager, including: a housing having a surface, a cold compress component, at least one massage component, and a control module;
a cold compress area is provided on the surface of the housing;
the cold compress component is provided in the housing and configured to provide cooling to the cold compress area;
the at least one massage component is provided in the housing and configured to massage breasts; and
the control module is provided in the housing and electrically connected to the cold compress component and the massage component respectively.

In an embodiment, the cold compress area is provided on a side surface of the housing.

In an embodiment, the cold compress component includes a semiconductor refrigeration piece, and a cold end of the semiconductor refrigeration piece is abutted against the cold compress area;

one side surface of the housing opposite to the cold compress area is provided with a heat dissipation hole corresponding to the semiconductor refrigeration piece; and

the cold compress component further includes a metal heat dissipation rack, one end of the metal heat dissipation rack is abutted against a hot end of the semiconductor refrigeration piece, and another end of the metal heat dissipation rack is provided with a heat dissipation fan.

In an embodiment, one side surface of the housing is configured as a massage surface, the massage surface includes the cold compress area, and the cold compress area is provided at one end of the massage surface.

In an embodiment, the massage component is a vibration massage component, and the vibration massage component includes a vibration motor provided in the housing.

In an embodiment, the massage component is provided at one end of the housing away from the cold compress component.

In an embodiment, the massage component is a kneading massage component, and the kneading massage component includes a kneading portion configured to knead breasts.

In an embodiment, the massage component is an electrical stimulation massage component, and the electrical stimulation massage component includes an electrical stimulation electrode configured to transmit an electrical stimulation to breasts.

In an embodiment, the massage surface further includes a warm compress area, the breast massager further includes a heating piece provided in the housing, and the heating piece is abutted against the warm compress area and is electrically connected to the control module.

In an embodiment, the housing is provided in a curved body structure, and the massage surface is provided on a concave side of the curved body structure.

In an embodiment, from a middle position of the curved body structure toward both ends of the curved body structure, a distance between the concave side of the curved body structure and a convex side of the curved body structure decreases, and one end of the curved body structure is configured as a pushing and scraping corner portion.

In an embodiment, the breast massager further includes a power module configured to supply power to the massage component, the cold compress component, and the control module;
the housing includes an inner housing and an outer housing sleeved on the inner housing;
the outer housing is configured as liquid silicone corresponding to the massage surface; and/or
the outer housing is configured as liquid silicone corresponding to the pushing and scraping corner portion; and/or
the outer housing is configured as a metal sheet corresponding to the cold compress area.

In the technical solution of the present application, the breast massager includes a housing, a cold compress component, at least one massage component and a control module. The side surface of the housing is provided with a cold compress area. When the breast congestion or swelling occurs, the control module controls the cold compress component to provide cooling to the cold compress area. After cooling, the cold compress area fits the breasts for cold compress. There is no need to take additional cold compress patches for freezing before nursing. The breast massager has a cold compress function, which can perform cold compress care on breasts, and the operation is convenient and quick, improving the user's experience.

### BRIEF DESCRIPTION OF THE DRAWINGS

In order to illustrate the technical solutions in the embodiments of the present application or in the related art more clearly, the following briefly introduces the accompanying drawings required for the description of the embodiments or the related art. Obviously, the drawings in the following description are only part of embodiments of the present application. For those skilled in the art, other drawings can also be obtained according to the structures shown in these drawings without any creative effort.

FIG. 1 is a schematic three-dimensional structural view of a breast massager according to an embodiment of the present application.

FIG. 2 is a schematic side view of the breast massager in FIG. 1.

FIG. 3 is a schematic cross-sectional view of the breast massager in FIG. 1.

FIG. 4 is a schematic bottom view of the breast massager in FIG. 1.

FIG. 5 is a module block view of the breast massager in FIG. 1.

The realization of the objective, functional characteristics, and advantages of the present application are further described with reference to the accompanying drawings.

### DETAILED DESCRIPTION OF THE EMBODIMENTS

The technical solutions of the embodiments of the present application will be described in more detail below with reference to the accompanying drawings. It is obvious that the embodiments to be described are only some rather than all of the embodiments of the present application. All other embodiments obtained by persons skilled in the art based on the embodiments of the present application without creative efforts shall fall within the scope of the present application.

It should be noted that all directional indications (such as up, down, left, right, front, back, etc.) in the embodiments of the present application are only used to explain the relative positional relationship, the movement situation, etc. among various assemblies under a certain posture as shown in the drawings. If the specific posture changes, the directional indication also changes accordingly.

In addition, the descriptions of “first”, “second”, etc. in the present application are only for the purpose of description, and should not be construed as indicating or implying relative importance or implicitly indicates the number of technical features indicated. Thus, a feature delimited with “first”, “second” may expressly or implicitly include at least one of that feature. In addition, the technical solutions between the various embodiments can be combined with each other, but must be based on the realization by those skilled in the art. When the combination of technical solutions is contradictory or cannot be realized, it should be considered that the combination of such technical solutions does not exist or fall within the scope of protection claimed in this application.

The main function of the existing breast massager is vibration massage to promote lactation and milk discharge, and solve problems such as milk stasis and mastitis for breastfeeding mothers. However, it cannot solve physiological breast congestion, swelling and other problems. At this time, breastfeeding mothers during pregnancy need to use cold compresses for breast care. The main material of cold compresses is gel, which needs to be frozen in the refrigerator before use, which is cumbersome to operate and wastes time.

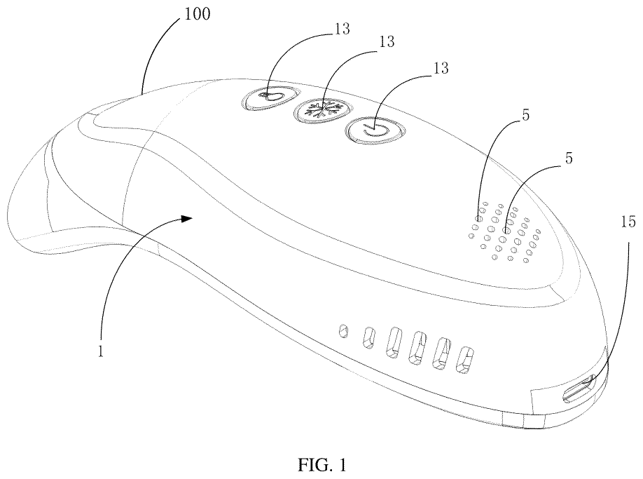

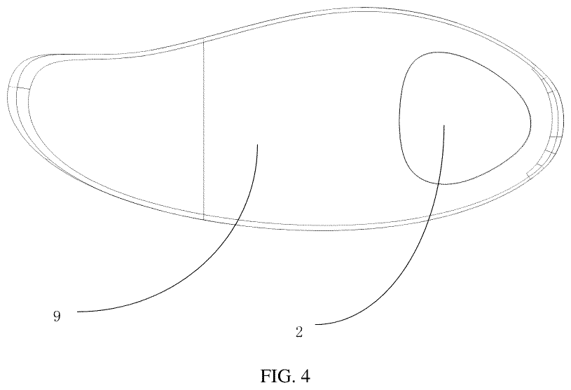

In view of this, the present application provides a breast massager which can perform cold compress care on breasts, and is easy and quick to operate, improving the user's experience. FIG. 1 to FIG. 5 are embodiments of the breast massager provided by the present application.

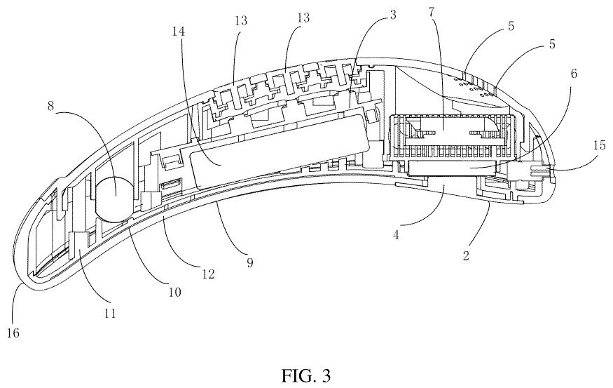

In an embodiment of the present application, referring to FIG. 1 to FIG. 5, with emphasis on FIG. 3, the breast massager 100 includes a housing 1, a cold compress component (see specifically the semiconductor refrigeration piece 4 in FIG. 3), at least one massage component (see the vibration motor 8 in the figure for details) and a control module 3. A side surface of the housing 1 is provided with a cold compress area 2; the massage component is provided in the housing 1 and is configured to perform massage operations on breasts; the cold compress component is provided in the housing 1 to provide cooling to the cold compress area 2; the control module 3 is provided in the housing 1, and the control module 3 is electrically connected to the cold compress component.

It should be noted that in addition to the side surface of the housing 1, the cold compress area 2 can also be provided on other surfaces or arc areas of the housing 1. In an embodiment, the cold compress area 2 is provided on at least one of the surfaces and arc areas of the housing 1, which is not limited in the present application.

In the technical solution of the present application, the breast massager 100 includes the housing 1, the cold compress component, at least one massage component and the control module 3. The side surface of the housing 1 is provided with the cold compress area 2. When breast congestion or swelling occurs, the control module 3 controls the cold compress component to provide cooling to the cold compress area 2. After cooling, the cold compress area 2 fits the breasts for cold compress, and there is no need to take another cold compress patch for freezing before nursing. The breast massager 100 has a cold compress function, can perform cold compress care on the breasts, and is easy and quick to operate, thereby improving the user's experience.

It should be supplemented that in the present application, the housing 1 is used as an installation carrier of the cold compress component, the massage component and the control module 3, and the specific structure of which is not limited. The housing 1 can be a cavity structure, which provides an installation space for the cold compress component, the massage component and the control module 3, and the side surface of the housing 1 refers to an outer side surface of a cavity wall of the cavity structure. The housing 1 can also be a space curved structure composed of a space curved template and edge components. The cold compress component, the massage component and the control module 3 are provided in an enclosed space of the space curved template. The side surface of the housing 1 can be the side surface of the space curved template facing away from the enclosed space, or a structural surface of the edge component. That is, regardless of the specific structure of the housing 1, the inside of the housing 1 only indicates that the housing 1 is used as the installation carrier and provides the installation space in which the cold compress component, the massage component and the control module 3 are provided. The side surface of the housing 1 refers to the structural surface of the housing 1 that can not be shielded by other components and can be exposed during the use of the product. The side surface is provided with the cold compress area 2 means that all or part of the side surface can be used to fit on breasts and apply cold compress to breasts, that is, the housing 1 can be regarded as a traditional cold compress, and the cooled cold compress area 2 is directly attached to breasts to cold compress on breasts.

The present application does not specifically limit the cooling method of the cold compress component. In this embodiment, the cold compress component includes a semiconductor refrigeration piece 4, and a cold end of the semiconductor refrigeration piece 4 is abutted against the cold compress area 2. A heat dissipation hole 5 is provided on the side surface of the housing 1 opposite to the cold compress area 2. The heat dissipation hole 5 is provided corresponding to the semiconductor refrigeration piece 4. The cold compress component also includes a metal heat dissipation rack 6. One end of the metal heat dissipation rack 6 is in abutted against the hot end of the semiconductor refrigeration piece 4, and the other end of the metal heat dissipation rack 6 is provided with a heat dissipation fan 7. The use of the semiconductor refrigeration piece 4 is conducive to miniaturization of the structure and is easy to install in the housing 1, no refrigerant is required, safe and pollution-free, no vibration, no noise, with fast cooling speed, and easy to control. The metal heat dissipation rack 6 conducts the heat from the hot end of the semiconductor refrigeration piece 4 to the heat dissipation fan 7 through the metal heat dissipation rack 6. After forced exhausting through the heat dissipation fan 7, the heat is finally discharged out of the housing 1 from the heat dissipation holes 5, which can prevent heat from accumulating in the housing 1, improve the heat dissipation efficiency, and help ensure the cooling effect of the semiconductor refrigeration piece 4, thereby ensuring the cold compress effect and improving the user's experience.

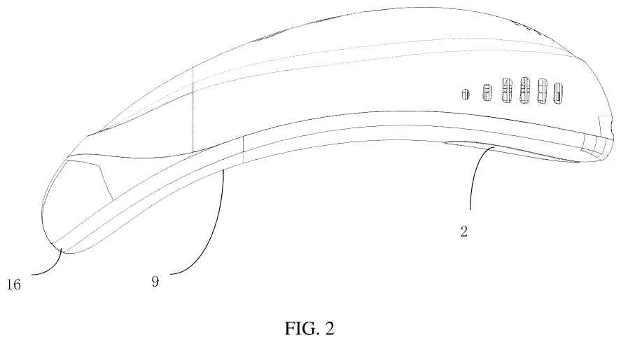

It can be understood that both the massage surface and the cold compress area 2 need to be configured according to the characteristics of breasts and have certain configuration requirements. In this embodiment, one side surface of the housing 1 is configured as a massage surface (the lower side surface in FIG. 2 and FIG. 3), the massage surface includes the cold compress area 2, and the cold compress area 2 is provided at one end of the massage surface. In this way, the side surface of the housing 1 is configured as the massage surface including the cold compress area 2, and the cold compress area 2 is part of the massage surface. The side surface can meet the requirements of massage and cold compress at the same time. Compared with separately setting the massage surface and the cold compress area 2, it can reduce the difficulty of overall product configuration and reduce product manufacturing costs. It should be supplemented that the massage surface refers to the structural surface of the housing 1 that is in contact with breasts during the use of the product. This structural surface can act on breasts to massage and stimulate breasts. The specific shape of the massage surface is not limited, as long as it meets the massage function requirements. It can be understood that the massage surface is a generally smooth surface, such as a flat surface, a hemispherical surface, a drop-shaped surface, and a smooth arc-shaped surface with a certain arc that matches the surface shape of breasts.

In this embodiment, the massage component is a vibration massage component. The vibration massage component includes a vibration motor 8. The vibration motor 8 is provided in the housing 1. It can be understood that the vibration motor 8 is provided in the housing 1, the vibration motor 8 can drive the breast massager 100 to vibrate as a whole, thereby vibrating breasts through the massage surface to massage the breasts and promote milk discharge.

In this embodiment, the massage component is provided at one end of the housing 1 away from the cold compress component. In this way, the mutual interference between the massage component and the cold compress component can be avoided, and the product structure is more reasonable and safer to use.

The specific form of the massage component is not limited in the present application. In an embodiment, the massage component is a kneading massage component, and the kneading massage component includes a kneading portion that can be used to knead breasts. In this way, it can produce a greater stimulation to breasts, relieve the pain, and achieve a better massage effects. Specifically, the kneading massage component may include a motor and a massage wheel. The massage wheel is rotatably mounted on the housing 1. The motor drives the massage wheel to rotate, so that the massage wheel forms the kneading portion. One side surface of the massage wheel is abutted against breasts and rolls on breasts to knead. The kneading massage component may also include a motor, a cam transmission mechanism and a deflection structure. The motor drives the cam transmission structure to rotate, thereby driving the deflection structure to deflect and swing. The deflection structure forms the kneading structure and is abutted against breasts and squeezes and rubs to knead. That is, the kneading massage component is a mechanical massage, and the kneading portion refers to a structure that can be displaced relative to the housing 1, and the kneading is realized by the squeezing and friction of the kneading portion on breasts.

In an embodiment, the massage component is an electrical stimulation massage component, and the electrical stimulation massage component includes an electrical stimulation electrode, and the electrical stimulation electrode can deliver the electrical stimulation to breasts. In this way, by applying a pulse current to breasts, the blood circulation of the breast is accelerated and the pain is relieved. The electrical stimulation can avoid mechanical damage to the breast skin, which is safer and more comfortable.

In order to occupy the breast massager 100 with a hot compress function, in this embodiment, the massage surface also includes a warm compress area 9, and the breast massager 100 also includes a heating piece 10 provided in the housing 1. The heating piece 10 is abutted against the warm compress area 9 and is electrically connected to the control module 3. In this way, the user can directly use the warm compress area 9 of the massage surface to apply a hot compress to breasts to help with the drainage of breasts, which solves the problem of breast congestion and improve the user's experience.

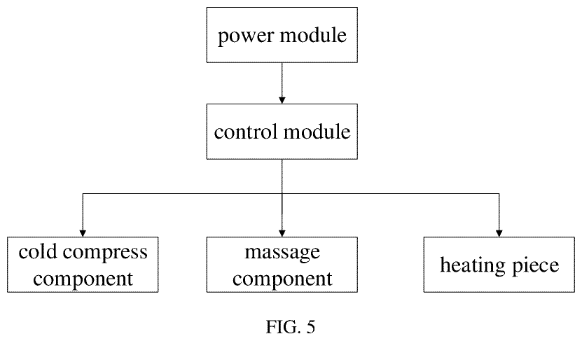

It should be pointed out that, as shown in FIG. 5, the heating piece 10, the massage component, and the cold compress component can be controlled independently, so that the hot compress, the massage, and the cold compress functions of the breast massager 100 can be used independently without interfering with each other. It can be understood that the side area of the housing 1 is limited, and the massage surface includes the cold compress area 2 and a warm compress area 9. The massage surface can satisfy the three functions of massage, cold compress, and hot compress. Compared with configuring the three functions separately, integrating three functions on one side surface can reduce the overall configuration difficulty of the product, leave more space for the configuration of other components of the product. For example, in an embodiment, the breast massager 100 is configured with a button 13 to facilitate users to make targeted selections. In addition, whether it is the massage surface, the cold compress area or the warm compress area, these three surfaces are all used closely with breasts. They have common features in the overall configuration requirements (such as smoothness). The three functions are integrated on one side surface, which also helps to reduce production costs.

In order to miniaturize and visualize the circuit and optimize the electrical layout, in an embodiment, the control module 3 is a printed circuit board (PCB).

In this embodiment, the housing 1 is provided in a curved body structure, and the massage surface is provided on the concave side of the curved body structure (the lower side in FIG. 2 and FIG. 3). On the one hand, the massage surface is made more suitable for the shape of breasts. On the other hand, the housing 1 with the curved structure can be held in hand or inserted between breasts and the underwear, which is more flexible to use.

Further, from a middle position of the curved body structure toward both ends of the curved body structure (the left and right ends in FIG. 2 and FIG. 3), a distance between the concave side of the curved body structure and the convex side of the curved body structure decreases, and one end of the curved body structure is configured as a pushing and scraping corner portion 16. In this way, the pushing and scraping corner portion 16 formed at one end of the curved body structure can occupy the breast massager 100 with a pushing and scraping function. When expelling milk, the user can hold the massager to push and scrape from the position of the mammary gland toward the nipple to promote lactation.

In this embodiment, referring to FIG. 3, the breast massager 100 also includes a power module (see battery 14). The power module supplied power for the massage component, the cold compress component, and the control module 3. The housing 1 includes an inner housing 11 and an outer housing 12 sleeved on the inner housing 11. In an embodiment, the outer housing 12 is configured to be made of liquid silicone corresponding to the massage surface. The liquid silicone material is soft, safe and non-toxic, which can ensure the safety of the product and improve the user's experience during massage. In an embodiment, the housing 12 is configured to be made of liquid silicone corresponding to the squeegee corner 16. The liquid silicone is soft, safe and non-toxic, which can ensure the safety of the product and improve the user's experience during pushing and scraping. In an embodiment, the outer housing 12 is configured as a metal sheet corresponding to the cold compress area 2, so as to ensure a cool feeling during cold compress. It can be understood that the metal sheet should be made of safe and non-toxic metal materials, such as stainless steel, aluminum alloy, etc. One, two, or all of the above three embodiments may exist at the same time. It can be understood that the best effect will be achieved if they exist at the same time. Specifically, the inner housing 11 and other parts of the outer housing 12 can be made of plastic to meet the strength and shape requirements.

In this embodiment, the power module is a battery 14, and the battery 14 supplies power to the massage component, the cold compress component, and the control module 3, so that the breast massager 100 can be used without being connected to power, which is more convenient. More specifically, the battery 14 is a rechargeable battery, and the breast massager 100 is provided with a charging port 15. The battery 14 is charged through the charging port 15. Compared with using disposable batteries, it is more economical and environmentally friendly, which can guarantee the electrical power and power supply.

The breast massager in the present application can be a comprehensive massager with cold compress function combined with at least one of the warm compress function, kneading function, vibration massage function, and electric stimulation massage function, and can provide users the massage or physical therapy with a variety of different functions. The breast massager in the present application can be used in conjunction with a breast pump product. For example, while the breast pump is sucking milk, the hand-held massager can apply physical therapy to the breast along the breast from side, top, and bottom of the breast to promote lactation, prevent milk blockage and relieve pain. In an embodiment, the product is roughly in the shape of a long strip. The user can hold the outer housing 12 to perform massage, warm compress or physical therapy on the breast. The corresponding physiotherapy structure is provided on the surface of one side of the outer housing 12, and the surface of the other side of the outer housing 12 is provided with function button, allowing users to automatically switch between function mode and gear mode according to needs.

The above descriptions are only embodiments of the present application, and are not intended to limit the scope of the present application. Under the inventive concept of the present application, any equivalent structural transformations made by using the contents of the description and drawings of the present application, or direct/indirect applications in other related technical fields are included in the scope of the present application.

1-12. (canceled)

13. A breast massager, comprising:
a housing, wherein one side surface of the housing is configured as a massage surface, the massage surface includes the cold compress area, and the cold compress area is an arc-shaped surface with a certain arc that matches a surface shape of breast;
a cold compress component provided in the housing and configured to provide cooling to the cold compress area;
at least one massage component provided in the housing and configured to massage breasts; and
a control module provided in the housing and electrically connected to the cold compress component and the massage component;
wherein the cold compress component and the massage component are capable of being used simultaneously;
the breast massager is configured with buttons to facilitate users to make targeted selections, and a surface of one side of the outer housing is provided with function buttons configured to be operated independently, so as to independently control the cold compress component and the massage component; and
the cold compress area and the at least one massage component are respectively provided at two ends of the housing.

14. The breast massager according to claim 13, wherein both the massage surface and the cold compress area are continuous smooth surfaces.

15. The breast massager according to claim 13, wherein the cold compress area is flush with the massage surface.

16. The breast massager according to claim 13, wherein the cold compress area is provided at one end of the massage surface.

17. The breast massager according to claim 13, wherein the massage component is a vibration massage component, and the vibration massage component comprises a vibration motor provided in the housing and at one end of the housing away from the cold compress area.

18. The breast massager according to claim 13, wherein the cold compress component comprises a semiconductor refrigeration piece, and a cold end of the semiconductor refrigeration piece is abutted against the cold compress area;
a heat dissipation hole is provided on the side surface of the housing opposite to the cold compress area; and
the heat dissipation hole is provided corresponding to the semiconductor refrigeration piece.

19. The breast massager according to claim 13, wherein the housing comprises an inner housing and an outer housing separated from the inner housing, and the outer housing is made of liquid silicone corresponding to the massage surface.
